(19) United States
(12) Patent Application Publication
(10) Pub. No.: US 2016/0303260 A1
(43) Pub. Date: Oct. 20, 2016

(54) 18F-Labelled Aldehyde Compositions for Radiofluorination

(71) Applicant: GE Healthcare Limited, Buckinghamshire (GB)

(72) Inventors: Torgrim Engell, Oslo (NO); Julian Grigg, Buckinghamshire (UK)

(73) Assignee: GE HEALTHCARE LIMITED, Buckinghamshire (UK)

(21) Appl. No.: 14/647,392

(22) PCT Filed: Nov. 25, 2013
(86) PCT No.: PCT/SE2013/074609

§ 371 Date: May 26, 2015

(30) Foreign Application Priority Data
Nov. 27, 2012 (GB) 1221266.8

(51) Int. Cl. A61K 51/08; A61K 49/00

(52) U.S. Cl. CPC A61K 51/088; A61K 49/0056

(57) ABSTRACT

The present invention relates to improved ¹⁸F-labelled aldehyde compositions, wherein impurities which affect imaging in vivo are identified and suppressed. Also provided are methods of preparation of radiofluorinated biological targeting molecules using said improved compositions, together with radiopharmaceutical compositions. The invention also includes methods of imaging and/or diagnosis using the radiopharmaceutical compositions described.

### FIELD OF THE INVENTION

The present invention relates to improved ¹⁸F-labelled aldehyde compositions, wherein impurities which affect imaging in vivo are identified and suppressed. Also provided are methods of preparation of radiofluorinated biological targeting molecules using said improved compositions, together with radiopharmaceutical compositions. The invention also includes methods of imaging and/or diagnosis using the radiopharmaceutical compositions described.

### BACKGROUND TO THE INVENTION

WO 2004/080492 discloses a method of radiofluorination of a vector which comprises reaction of a compound of formula (I) with a compound of formula (II):

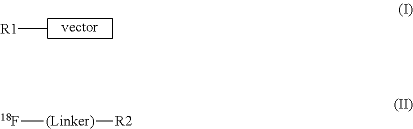

or a compound of formula (III) with a compound of formula (IV)

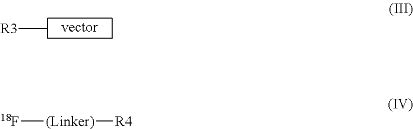

wherein:
R1 is an aldehyde moiety, a ketone moiety, a protected aldehyde such as an acetal, a protected ketone, such as a ketal, or a functionality, such as diol or N-terminal serine residue, which can be rapidly and efficiently oxidised to an aldehyde or ketone using an oxidising agent;
R2 is a group selected from primary amine, secondary amine, hydroxylamine, hydrazine, hydrazide, aminoxy, phenylhydrazine, semicarbazide, and thiosemicarbazide and is preferably a hydrazine, hydrazide or aminoxy group;
R3 is a group selected from primary amine, secondary amine, hydroxylamine, hydrazine, hydrazide, aminoxy, phenylhydrazine, semicarbazide, or thiosemicarbazide, and is preferably a hydrazine, hydrazide or aminoxy group;
R4 is an aldehyde moiety, a ketone moiety, a protected aldehyde such as an acetal, a protected ketone, such as a ketal, or a functionality, such as diol or N-terminal serine residue, which can be rapidly and efficiently oxidised to an aldehyde or ketone using an oxidising agent;
   to give a conjugate of formula (V) or (VI) respectively:

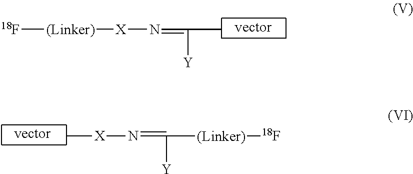

wherein X is —CO—NH—, —NH—, —O—, —NHCONH—, or —NHCSNH—, and is

preferably —CO—NH—, —NH— or —O—; Y is H, alkyl or aryl substituents; and the Linker group in the formulae (II), (IV), (V) and (VI) is selected from:

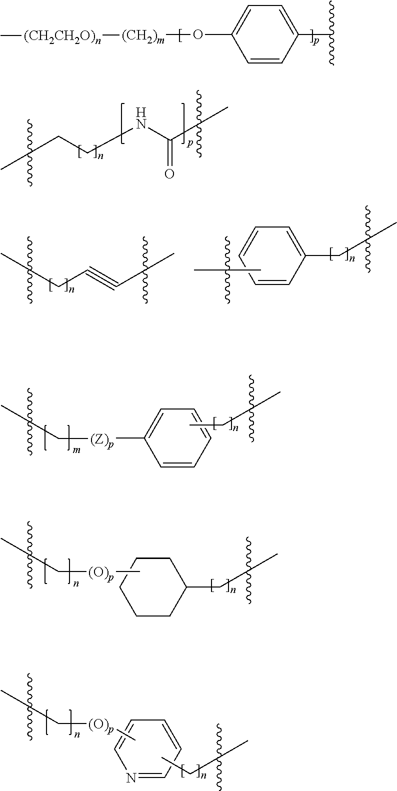

wherein n is an integer of 0 to 20; m is an integer of 1 to 10; p is an integer of 0 or 1; Z is O or S.
WO 2006/030291 discloses a method for radiofluorination comprising reaction of a compound of formula (I):

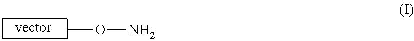

wherein the vector comprises the fragment:

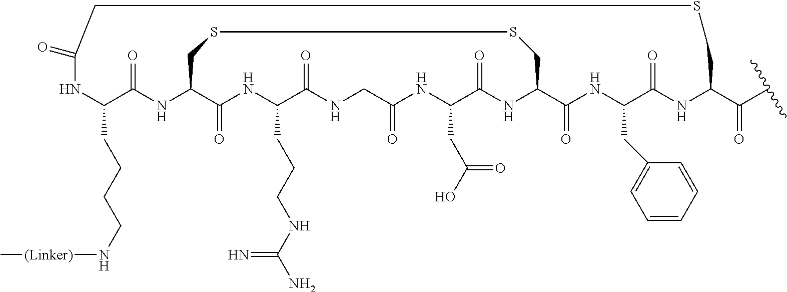

with a compound of formula (II):

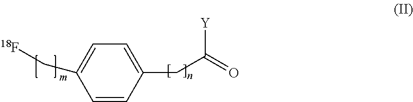

wherein:
n is an integer of 0 to 20;
m is an integer of 0 to 10;
Y is hydrogen, C₁₋₆alkyl, or phenyl
to give a compound of formula (III):

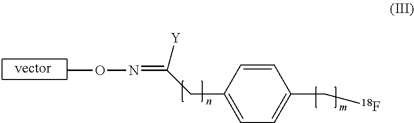

wherein m, n, and Y are defined as for the compound of formula (II) and the vector is as defined for the compound of formula (I).

Glaser et al [Bioconj.Chem., 19(4), 951-957 (2008)] describe the synthesis of ¹⁸F-labelled aldehydes, including ¹⁸F-fluorobenzaldehyde, and their conjugation to amino-oxy functionalised cyclic RGD peptides.

Battle et al [J.Nucl.Med., 52(3), 424-430 (2011)] disclose monitoring anti-angiogenic therapy with [¹⁸F]-fluciclatide:

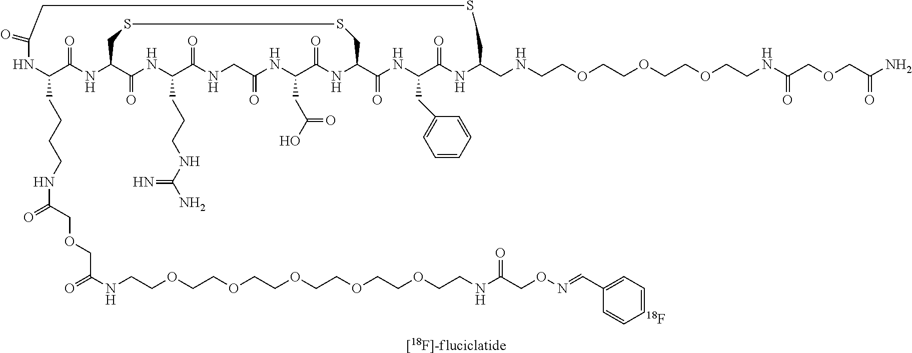

WO 2012/089594 discloses a method of preparation of¹⁸F-labelled fluoride ion (¹⁸F) for use in a radiofluorination reaction, which employs an improved eluent to elute the ¹⁸F-labelled fluoride ion from an ion exchange resin. The method comprises:
(i) trapping an aqueous solution of ¹⁸F⁻ onto an ion exchange column; and,
(ii) passing an eluent solution through said ion exchange column on which said ¹⁸F⁻ is adsorbed to obtain an ¹⁸F⁻ eluent,
wherein said eluent solution comprises a cationic counterion in a suitable solvent with the proviso that said eluent solution does not comprise acetonitrile.

WO 2012/089594 teaches that, when the eluent solution contains acetonitrile, the acetonitrile can hydrolyse on standing forming acetamide and ammonium acetate, and those impurities can cause radiochemical purity problems when using the eluted ¹⁸F⁻ in subsequent ¹⁸F radiolabelling reactions.

The present inventors have, however, found that the conjugation of ¹⁸F-labelled aldehydes (such as ¹⁸F-fluorobenzaldehyde or FBA) with functionalised peptides suffers from unexpected limitations in both radiochemical purity and yield. There is therefore still a need for alternative methods of labelling biological targeting moieties with ¹⁸F.

### The Present Invention.

The present invention provides improved ¹⁸F-labelled aldehyde compositions, and their application to the radiofluorination of a biological targeting moiety (BTM). Improved radiopharmaceutical compositions derived from the conjugation of ¹⁸F-labelled aldehydes to aminooxy- or amine- functionalised BTMs are also provided.

The invention is based on detailed analyses of the different chemical species present in such aldehydes, and an understanding of how they may be carried through into the radiolabelled BTM product—plus how best to suppress the impurity species. The cyanovinyl compounds were not recognised in the prior art, yet can arise even when minute traces of acetonitrile are present. The higher radiochemical purity and yield facilitates more robust manufacture for clinical use, as well as suppression of unnecessary radiation dose to the patient.

In addition, the improved radiopharmaceutical compositions of the present invention can be achieved in shorter preparation times, which minimises any loss of ¹⁸F (half-life 109 minutes) radioactive content during the preparation and purification steps prior to use. The compositions of the present invention can be obtained using methodology which is amenable to automation on a commercial automated synthesizer apparatus—an advantage over prior art HPLC methods (which cannot be automated in this way). Automation confers improved reproducibility, as well as reduced operator radiation dose.

Furthermore, the higher radiochemical yield and purity of the product means that less functionalised BTM needs to be used to obtain the same amount of radioactive product. Since the unlabelled BTM will compete for the same biological site in vivo, lowering the amount of functionalised BTM present helps preserve the efficacy of the radiolabelled product. In addition, since the BTM may be e.g. a complex polypeptide or protein which is expensive and time-consuming to obtain, that is an important efficiency of time/materials.

### DETAILED DESCRIPTION OF THE INVENTION

The present inventors have found that the preparation of ¹⁸F-fluorobenzaldehyde (FBA) suffers from previously unrecognized problems when traces of acetonitrile are present (Scheme 1):

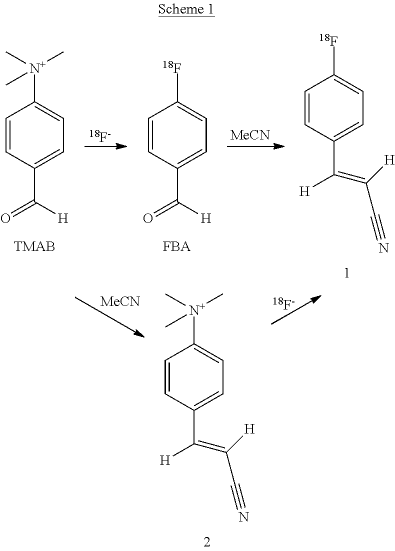

As acetonitrile reacts with both ¹⁸F-FBA (the desired product) and TMAB (the precursor), the cyanovinyl product (1) continues to be produced, even when the TMAB is consumed. The rate constant for the radiofluorination of TMAB is believed to be higher than that for cyanovinyl formation from TMAB, because FBA is formed in the presence of acetonitrile. The rate of FBA formation slows down as fluoride is consumed, i.e. the concentration of fluoride decreases. The opposite will be the case for the slower cyanovinyl formation from FBA—the rate will increase as the FBA concentration increases. Hence, at a given point the rate of cyanovinyl formation by reaction between FBA and acetonitrile will be higher than the rate of fluorination. Unfortunately, the conditions which increase the rate of fluorination also appear to favour cyanovinyl formation. The fact that the cyanovinyl product (1) may also occur via the intermediate (2) enhances the negative effect of acetonitrile. The consequence is a limitation in the yield of ¹⁸F-FBA caused by the presence of acetonitrile—which applicants found to be no more than ca. 60%.

A further complication is that any other benzaldehyde species present in the reaction mixture, such as DMAB (4-dimethylaminobenzaledehyde), also react with acetonitrile to give further cyanovinyl impurities.

At the start of the TMAB radiofluorination reaction, the ¹⁹F-fluoride content (which corresponds to the overall fluoride chemical content) is less than 1 μg, typically 0.1 to 0.5 μg. The presence of only 2 μg or less of acetonitrile is thus needed to reach one mole equivalent to fluoride.

The present inventors have found that the cyanovinyl adduct forms readily from an ¹⁸F-labelled aldehyde and acetonitrile, especially under basic conditions and at temperatures above room temperature, ca. 50-70° C. The ¹⁸F-labelled aldehyde typically requires such reaction conditions for both radiosynthesis, and subsequent conjugation reactions. Hence, such cyanovinyl impurities may be generated whenever an ¹⁸F-labelled aldehyde is exposed to acetonitrile, even in trace quantities. The problem is that such reaction conditions are exactly those necessary to achieve satisfactory yields in both the radiosynthesis and conjugation reactions.

In order to address the above problems, in a first aspect, the present invention provides an ¹⁸F-labelled aldehyde composition which comprises an ¹⁸F-labelled aldehyde of Formula (I) and an ¹⁸F-labelled vinyl cyanide of Formula (II):

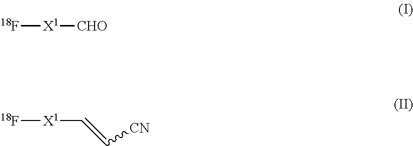

where X¹ is the same in Formulae (I) and (II), and is a C₄₋₁₆ bivalent organic radical;
and wherein (i) the molar ratio of I:II is at least 10:1; and
   (ii) acetonitrile is excluded from said composition.

The term “composition” has its conventional meaning and refers to a mixture of the radiofluorinated aldehyde of Formula (I) with the ¹⁸F-labelled vinyl cyanide of Formula (II). The composition is suitably in solution.

In Formula II, as well as IIA-IID, the wavy bond denotes that the stereochemistry at the C═C double bond is undefined—i.e. that either diastereoisomer (E or Z) can be present, depending on whether the cyano group is cis or trans to X¹. The present invention encompasses mixtures of such isomers, as well as mixtures enriched in one such diastereomer, as well as pure diastereomers.

The term “¹⁸F-labelled” has its conventional meaning in the field of PET radiotracers, and implies that the fluorine substituent has an elevated or enriched level of the radioisotope ¹⁸F when compared to normal isotopic abundance. Such elevation is typically such that both the radioactive dose and radioactive concentration are suitable for in vivo imaging.

By the term “C₄₋₁₆ bivalent organic radical” is meant a substituted or unsubstituted organic radical which may comprises one or more of the following (or combinations thereof): arylene ring; heteroarylene ring, an alkylene chain and optionally 1 to 5 heteroatoms independently chosen from O, N and S. When two or more heteroatoms are incorporated, the bivalent organic radical excludes direct heteroatom-heteroatom bonds. Preferably, the bivalent organic radical comprises at least one aryl or heteroaryl ring, more preferably one such ring.

By the term “acetonitrile is excluded” is meant that the composition, in particular any solvents used, or any of the reactants/precursors used to prepare the radiofluorinated aldehyde in situ do not comprise acetonitrile. It is particularly important to use longer drying times and preferably higher vacuum in order to remove traces of acetonitrile when drying the [¹⁸F]-fluoride. In addition, special steps are appropriate to remove any traces of acetonitrile that could be present as a residual solvent as a result of purification and/or chromatography carried out on said reactants/precursors. That is because the present inventors have established that acetonitrile can react with radiofluorinated aldehydes to give the vinyl cyanide compounds of Formula II.

The terms “comprising” or “comprises” have their conventional meaning throughout this application and imply that the composition must have the components listed, but that other, unspecified compounds or species may be present in addition. The terms therefore include as a preferred subset “consisting essentially of” which means that the composition has the components listed without other compounds or species being present.

### Preferred Embodiments.

In the first aspect, the molar ratio of I:II is preferably at least 20:1, more preferably at least 30:1, most preferably at least 100:1.

The ¹⁸F-labelled aldehyde composition of the first aspect is preferably chosen such that the ¹⁸F-labelled aldehyde is of Formula (IA) and the ¹⁸F-labelled vinyl cyanide is of Formula (IIA):

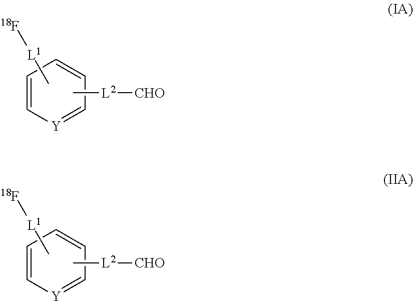

where:
Y is independently C or N;
L¹ and L² are independently linker groups chosen from —(CH₂)ₓ—, —O—(CH₂)_{y}— or —(OCH₂CH₂)_{y}—;
   wherein x is independently an integer of value 0 to 3, and
   y is independently an integer of value 2 to 4.

In Formulae IA and IIA, linker groups L¹ and L² are located at two different positions of the aryl ring. When Y is N, L¹ and L² are located at two different positions other than Y.

In Formulae IA and IIA, L² is preferably —(CH₂)ₓ— with x=0, such that the aldehyde group of IA is directly bonded to the aryl ring.

In Formulae IA and IIA, Y is preferably C. A preferred such embodiment is when the ¹⁸F-labelled aldehyde is of Formula (IB) and the ¹⁸F-labelled vinyl cyanide is of Formula (IIB):

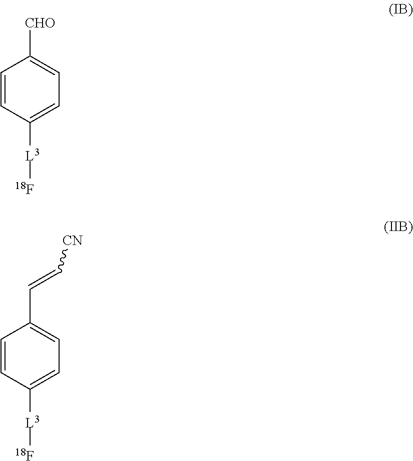

where L³ is —(CH₂)ₓ—or —O—(CH₂)_{y}—, and x and y are as defined for IA and IIA.

A more preferred embodiment is when the ¹⁸F-labelled aldehyde is of Formula (IC) and the ¹⁸F-labelled vinyl cyanide is of Formula (IIC):

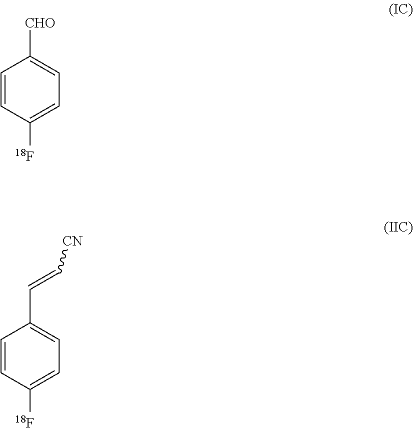 [SMILES: O=Cc1ccc([18F])cc1 ; N#CC=Cc1ccc([18F])cc1]

In Formulae IA and IIA, when Y is N, a preferred such embodiment is where the ¹⁸F-labelled aldehyde is of Formula (ID) and the ¹⁸F-labelled vinyl cyanide is of Formula (IID):

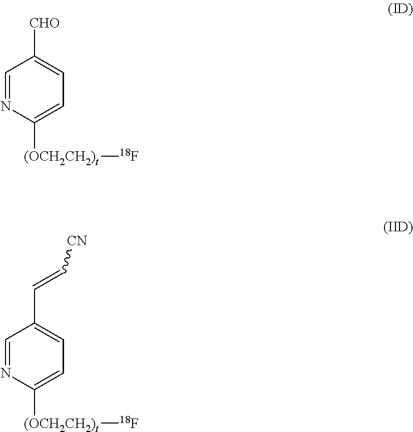

wherein t is an integer of value 1 to 3.

The ¹⁸F-labelled aldehyde composition of the first aspect is preferably provided in solution in a water miscible organic solvent or an aqueous mixture thereof. The “water miscible organic solvent” excludes acetonitrile, but is preferably chosen from a solvent having a boiling point of less than 80° C., more preferably less than 70° C. Suitable such solvents are designed to have minimal reactivity with the aldehyde group of the aldehyde of Formula (I), and include: methanol, ethanol, tetrahydrofuran or aqueous mixtures thereof. More preferably, the solvent is methanol, ethanol or aqueous mixtures thereof. Most preferably, the solvent is ethanol or aqueous ethanol.

In a preferred embodiment, the ¹⁸F-labelled aldehyde composition of the first aspect is provided as a radiopharmaceutical composition which comprises the ¹⁸F-labelled aldehyde composition together with a biocompatible carrier, in a form suitable for mammalian administration.

By the phrase “in a form suitable for mammalian administration” is meant a composition which is sterile, pyrogen-free, lacks compounds which produce toxic or adverse effects, and is formulated at a biocompatible pH (approximately pH 4.0 to 10.5). Such compositions lack particulates which could risk causing emboli in vivo, and arc formulated so that precipitation does not occur on contact with biological fluids (e.g. blood). Such compositions also contain only biologically compatible excipients, and are preferably isotonic.

The “biocompatible carrier” is a fluid, especially a liquid, in which the imaging agent can be suspended or preferably dissolved, such that the composition is physiologically tolerable, i.e. can be administered to the mammalian body without toxicity or undue discomfort. The biocompatible carrier is suitably an injectable carrier liquid such as sterile, pyrogen-free water for injection; an aqueous solution such as saline (which may advantageously be balanced so that the final product for injection is isotonic); an aqueous buffer solution comprising a biocompatible buffering agent (e.g. phosphate buffer); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). Preferably the biocompatible carrier is pyrogen-free water for injection, isotonic saline or phosphate buffer.

The radiopharmaceutical composition is supplied in a suitable vial or vessel which comprises a sealed container which permits maintenance of sterile integrity and/or radioactive safety, plus optionally an inert headspace gas (e.g. nitrogen or argon), whilst permitting addition and withdrawal of solutions by syringe or cannula. A preferred such container is a septum-sealed vial, wherein the gas-tight closure is crimped on with an overseal (typically of aluminium). The closure is suitable for single or multiple puncturing with a hypodermic needle (e.g. a crimped-on septum seal closure) whilst maintaining sterile integrity. Such containers have the additional advantage that the closure can withstand vacuum if desired (e.g. to change the headspace gas or degas solutions), and withstand pressure changes such as reductions in pressure without permitting ingress of external atmospheric gases, such as oxygen or water vapour.

Preferred multiple dose containers comprise a single bulk vial which contains multiple patient doses, whereby single patient doses can thus be withdrawn into clinical grade syringes at various time intervals during the viable lifetime of the preparation to suit the clinical situation. Pre-filled syringes are designed to contain a single human dose, or “unit dose” and are therefore preferably a disposable or other syringe suitable for clinical use. The pharmaceutical compositions of the present invention preferably have a dosage suitable for a single patient and are provided in a suitable syringe or container, as described above.

The pharmaceutical composition may contain additional optional excipients such as: an antimicrobial preservative, pH-adjusting agent, filler, radioprotectant, solubiliser or osmolality adjusting agent. By the term “radioprotectant” is meant a compound which inhibits degradation reactions, such as redox processes, by trapping highly-reactive free radicals, such as oxygen-containing free radicals arising from the radiolysis of water. The radioprotectants of the present invention are suitably chosen from: ascorbic acid, para-aminobenzoic acid (i.e. 4-aminobenzoic acid), gentisic acid (i.e. 2,5-dihydroxybenzoic acid) and salts thereof with a biocompatible cation. By the term “biocompatible cation” (B^{c}) is meant a positively charged counterion which forms a salt with an ionised, negatively charged group, where said positively charged counterion is also non-toxic and hence suitable for administration to the mammalian body, especially the human body. Examples of suitable biocompatible cations include: the alkali metals sodium or potassium; the alkaline earth metals calcium and magnesium; and the ammonium ion. Preferred biocompatible cations are sodium and potassium, most preferably sodium.

By the term “solubiliser” is meant an additive present in the composition which increases solubility in the solvent. A preferred such solvent is aqueous media, and hence the solubiliser preferably improves solubility in water. Suitable such solubilisers include: C₁₋₄ alcohols; glycerine; polyethylene glycol (PEG); propylene glycol; polyoxyethylene sorbitan monooleate; sorbitan monooloeate; polysorbates; poly(oxyethylene)poly(oxypropylene)poly(oxyethylene) block copolymers (Pluronics™); cyclodextrins (e.g. alpha, beta or gamma cyclodextrin, hydroxypropyl-β-cyclodextrin or hydroxypropyl-γ-cyclodextrin) and lecithin.

By the term “antimicrobial preservative” is meant an agent which inhibits the growth of potentially harmful micro-organisms such as bacteria, yeasts or moulds. The antimicrobial preservative may also exhibit some bactericidal properties, depending on the dosage employed. The main role of the antimicrobial preservative(s) of the present invention is to inhibit the growth of any such micro-organism in the pharmaceutical composition. The antimicrobial preservative may, however, also optionally be used to inhibit the growth of potentially harmful micro-organisms in one or more components of kits used to prepare said composition prior to administration. Suitable antimicrobial preservative(s) include: the parabens, i.e. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the composition is within acceptable limits (approximately pH 4.0 to 10.5) for human or mammalian administration. Suitable such pH-adjusting agents include pharmaceutically acceptable buffers, such as tricine, phosphate, citrate or TRIS [i.e. tris(hydroxymethyl)aminomethane], and pharmaceutically acceptable bases such as sodium carbonate, sodium bicarbonate or mixtures thereof. When the composition is employed in kit form, the pH adjusting agent may optionally be provided in a separate vial or container, so that the user of the kit can adjust the pH as part of a multi-step procedure.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

The radiopharmaceutical compositions may be prepared under aseptic manufacture (i.e. clean room) conditions to give the desired sterile, non-pyrogenic product. It is preferred that the key components, especially the associated reagents plus those parts of the apparatus which come into contact with the imaging agent (e.g. vials) are sterile. The components and reagents can be sterilised by methods known in the art, including: sterile filtration, terminal sterilisation using e.g. gamma-irradiation, autoclaving, dry heat or chemical treatment (e.g. with ethylene oxide). It is preferred to sterilise some components in advance, so that the minimum number of manipulations needs to be carried out. As a precaution, however, it is preferred to include at least a sterile filtration step as the final step in the preparation of the pharmaceutical composition.

The ¹⁸F-labelled aldehyde compositions of the first aspect can be obtained by one or more of the following:
(i) for ¹⁸F-fluoride used in the aldehyde radiosynthesis, ensuring that steps to dry ¹⁸F-fluoride ion to remove acetonitrile are carried out rigorously and/or multiple times and/or under higher vacuum to remove even microgramme quantities of acetonitrile;
(ii) use of solvent(s) in the radiofluorination reaction wherein the ¹⁸F-labelled aldehyde is prepared that are of high purity and have particularly low levels of acetonitrile;
(iii) formulation of the ¹⁸F-labelled aldehyde, once prepared, in a suitable water miscible organic solvent which excludes acetonitrile (as described above);
(v) chromatography techniques such as SPE (solid phase extraction) using solvents other than acetonitrile, to separate cyanovinyl species if present.

In a second aspect, the present invention provides a method of ¹⁸F-radiolabelling a biological targeting molecule, which comprises:
(i) provision of the ¹⁸F-labelled aldehyde composition of claim 1;
(ii) provision of a functionalised biological targeting molecule of Formula III:

Y¹-[BTM] (III)
wherein Y¹ is —NH₂ or —O—NH₂;
(iii) reaction of the composition from step (i) with Y¹-[BTM] from step (ii) to give the ¹⁸F-radiolabelled biological targeting molecule of Formula IV:

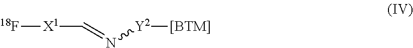

wherein Y² is absent or is —O—.

Preferred embodiments of ¹⁸F-labelled aldehyde composition in the second aspect are as described in the first aspect (above).

By the term “biological targeting moiety” (BTM) is meant a compound which, after administration, is taken up selectively or localises at a particular site of the mammalian body in vivo. Such sites may be implicated in a particular disease state or be indicative of how an organ or metabolic process is functioning.

By the term “functionalised biological targeting molecule” is meant that the BTM either already comprises an amine or aminooxy functional group, or has been derivatised to covalently attach an amine or aminooxy functional group. The term “aminooxy group” means that the BTM has covalently conjugated thereto an aminooxy functional group. Such groups are of formula —O—NH₂, preferably —CH₂O—NH₂ and have the advantage that the amine of the amino-oxy group is more reactive than a Lys amine group in condensation reactions with aldehydes to form oxime ethers having the linkage C═N—O—C. Hence, Y¹ is preferably —O—NH₂.

The radiofluorinated BTM of Formula (IV) is preferably a radiotracer imaging agent. By the term “imaging agent” is meant a compound suitable for imaging the mammalian body. Preferably, the mammal is an intact mammalian body in vivo, and is more preferably a human subject. Preferably, the imaging agent can be administered to the mammalian body in a minimally invasive manner, i.e. without a substantial health risk to the mammalian subject when carried out under professional medical expertise. Such minimally invasive administration is preferably intravenous administration into a peripheral vein of said subject, without the need for local or general anaesthetic. The imaging agent is designed and administered at a dosage suitable to have the minimal pharmacological effect—so that it is as representative as possible of the status of the mammalian body.

The term “in vivo imaging” as used herein refers to those techniques that non-invasively produce images of all or part of an internal aspect of a mammalian subject. A preferred imaging technique of the present invention is positron emission tomography (PET).

The method of the second aspect is suitable carried out in solution.

The BTM preferably comprises: a 3-80 mer peptide, peptide analogue, peptoid or peptide mimetic which may be a linear or cyclic peptide or combination thereof; a single amino acid; an enzyme substrate, enzyme antagonist, enzyme agonist (including partial agonist) or enzyme inhibitor; receptor-binding compound (including a receptor substrate, antagonist, agonist or substrate); oligonucleotides, or oligo-DNA or oligo-RNA fragments.

By the term “amino acid” is meant an L- or D-amino acid, amino acid analogue (eg. naphthylalanine) which may be naturally occurring or of purely synthetic origin, and may be optically pure, i.e. a single enantiomer and hence chiral, or a mixture of enantiomers. Conventional 3-letter or single letter abbreviations for amino acids are used herein. Preferably the amino acids of the present invention are optically pure.

By the term “peptide” is meant a compound comprising two or more amino acids, as defined below, linked by a peptide bond (i.e. an amide bond linking the amine of one amino acid to the carboxyl of another). The term “peptide mimetic” or “mimetic” refers to biologically active compounds that mimic the biological activity of a peptide or a protein but are no longer peptidic in chemical nature, that is, they no longer contain any peptide bonds (that is, amide bonds between amino acids). Here, the term peptide mimetic is used in a broader sense to include molecules that are no longer completely peptidic in nature, such as pseudo-peptides, semi-peptides and peptoids. The term “peptide analogue” refers to peptides comprising one or more amino acid analogues, as described below. See also Synthesis of Peptides and Peptidomimetics, M. Goodman et al, Houben-Weyl Vol E22c of Methods in Organic Chemistry, Thieme (2004).

By the term “sugar” is meant a mono-, di- or tri- saccharide. Suitable sugars include: glucose, galactose, maltose, mannose, and lactose. Optionally, the sugar may be functionalised to permit facile coupling to amino acids. Thus, e.g. a glucosamine derivative of an amino acid can be conjugated to other amino acids via peptide bonds. The glucosamine derivative of asparagine (commercially available from NovaBiochem) is one example of this:

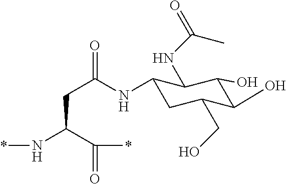 [SMILES: *N[C@@H](CC(=O)NC1CC(CO)C(O)C(O)C1NC(C)=O)C(*)=O]

The term “polyethyleneglycol polymer” or “PEG” has its conventional meaning, as described e.g. in “The Merck Index”, 14th Edition entry 7568, i.e. a liquid or solid polymer of general formula H(OCH₂CH₂)ₙOH where n is an integer greater than or equal to 4. The polyethyleneglycol polymers of the present invention may be linear or branched, but are preferably linear. The polymers are also preferably non-dendrimeric. Preferred PEG-containing linker groups comprise units derived from oligomerisation of the monodisperse PEG-like structures of Formulae Bio1 or Bio2:

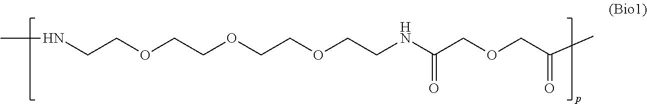

17-amino-5-oxo-6-aza-3, 9, 12, 15-tetraoxaheptadecanoic acid of Formula Bio1 wherein p is an integer from 1 to 10. Alternatively, a PEG-like structure based on a propionic acid derivative of Formula Bio2 can be used:

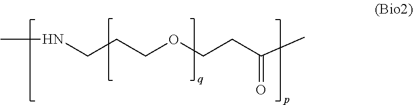

where p is as defined for Formula Bio1 and q is an integer from 3 to 15. In Formula Bio2, p is preferably 1 or 2, and q is preferably 5 to 12.

### Preferred embodiments

The BTM may be of synthetic or natural origin, but is preferably synthetic. The term “synthetic” has its conventional meaning, i.e. man-made as opposed to being isolated from natural sources eg. from the mammalian body. Such compounds have the advantage that their manufacture and impurity profile can be fully controlled. Monoclonal antibodies and fragments thereof of natural origin are therefore outside the scope of the term ‘synthetic’ as used herein. The BTM is preferably non-proteinaceous, i.e. does not comprise a protein.

The molecular weight of the BTM is preferably up to 15,000 Daltons. More preferably, the molecular weight is in the range 200 to 12,000 Daltons, most preferably 300 to 10,000 Daltons, with 400 to 9,000 Daltons being especially preferred. When the BTM is a non-peptide, the molecular weight of the BTM is preferably up to 3,000 Daltons, more preferably 200 to 2,500 Daltons, most preferably 300 to 2,000 Daltons, with 400 to 1,500 Daltons being especially preferred.

When the BTM is an enzyme substrate, enzyme antagonist, enzyme agonist, enzyme inhibitor or receptor-binding compound it is preferably a non-peptide, and more preferably is synthetic. By the term “non-peptide” is meant a compound which does not comprise any peptide bonds, i.e. an amide bond between two amino acid residues. Suitable enzyme substrates, antagonists, agonists or inhibitors include glucose and glucose analogues such as fluorodeoxyglucose; fatty acids, or elastase, Angiotensin II or metalloproteinase inhibitors. A preferred non-peptide Angiotensin II antagonist is Losartan. Suitable synthetic receptor-binding compounds include estradiol, estrogen, progestin, progesterone and other steroid hormones; ligands for the dopamine D-1 or D-2 receptor, or dopamine transporter such as tropanes; and ligands for the serotonin receptor.

The BTM is most preferably a 3-100 mer peptide or peptide analogue. When the BTM is a peptide, it is preferably a 4-30 mer peptide, and most preferably a 5 to 28-mer peptide. When the BTM is a peptide, preferred such peptides include:
somatostatin, octreotide and analogues,
peptides which bind to the ST receptor, where ST refers to the heat-stable toxin produced by E.coli and other micro-organisms;
bombesin;
vasoactive intestinal peptide;
neurotensin;
laminin fragments eg. YIGSR, PDSGR, IKVAV, LRE and KCQAGTFALRGDPQG,
N-formyl chemotactic peptides for targeting sites of leucocyte accumulation,
Platelet factor 4 (PF4) and fragments thereof,

RGD (Arg-Gly-Asp)-containing peptides, which may eg. target angiogenesis [R.Pasqualini et al., Nat Biotechnol. 1997 Jun;15(6):542-6]; [E. Ruoslahti, Kidney Int. 1997 May;51(5):1413-7].
peptide fragments of a₂-antiplasmin, fibronectin or beta-casein, fibrinogen or thrombospondin. The amino acid sequences of α₂-antiplasmin, fibronectin, beta-casein, fibrinogen and thrombospondin can be found in the following references: α₂-antiplasmin precursor [M.Tone et al., J.Biochem, 102, 1033, (1987)]; beta-casein [L.Hansson et al, Gene, 139, 193, (1994)]; fibronectin [A.Gutman et al, FEBS Lett., 207, 145, (1996)]; thrombospondin-1 precursor [V.Dixit et al, Proc. Natl. Acad. Sci., USA, 83, 5449, (1986)]; R.F.Doolittle, Ann. Rev. Biochem., 53, 195, (1984);
peptides which are substrates or inhibitors of angiotensin, such as: angiotensin II Asp-Arg-Val-Tyr-Ile-His-Pro-Phe (E. C. Jorgensen et al, J. Med. Chem., 1979, Vol 22, 9, 1038-1044) [Sar, Ile] Angiotensin II: Sar-Arg-Val-Tyr-Ile-His-Pro-Ile (R.K. Turker et al., Science, 1972, 177, 1203).
Angiotensin I: Asp-Arg-Val-Tyr-Ile-His-Pro-Phe-His-Leu;
c-Met targeting peptides.

When the BTM is a peptide, one or both termini of the peptide, preferably both, have conjugated thereto a metabolism inhibiting group (M^{IG}). Having both peptide termini protected in this way is important for in vivo imaging applications, since otherwise rapid metabolism would be expected with consequent loss of selective binding affinity for the BTM peptide. By the term “metabolism inhibiting group” (M^{IG}) is meant a biocompatible group which inhibits or suppresses enzyme, especially peptidase such as carboxypeptidase, metabolism of the BTM peptide at either the amino terminus or carboxy terminus. Such groups are particularly important for in vivo applications, and are well known to those skilled in the art and are suitably chosen from, for the peptide amine terminus:

N-acylated groups —NH(C═O)R^{G} where the acyl group —(C═O)R^{G} has R^{G} chosen from: C₁₋₆ alkyl, C₃₋₁₀ aryl groups or comprises a polyethyleneglycol (PEG) building block. Suitable PEG groups are described for the linker group (L¹), above. Preferred such PEG groups are the biomodifiers of Formulae Bio1 or Bio2 (above). Preferred such amino terminus M^{IG} groups are acetyl, benzyloxycarbonyl or trifluoroacetyl, most preferably acetyl.

Suitable metabolism inhibiting groups for the peptide carboxyl terminus include: carboxamide, tert-butyl ester, benzyl ester, cyclohexyl ester, amino alcohol or a polyethyleneglycol (PEG) building block. A suitable M^{IG} group for the carboxy terminal amino acid residue of the BTM peptide is where the terminal amine of the amino acid residue is N-alkylated with a C₁₋₄ alkyl group, preferably a methyl group.

Preferred such M^{IG} groups are carboxamide or PEG, most preferred such groups are carboxamide.

Preferred BTM peptides are RGD peptides or c-Met targeting peptides. A most preferred such RGD peptide is when the BTM is a peptide of Formula (BTM1):

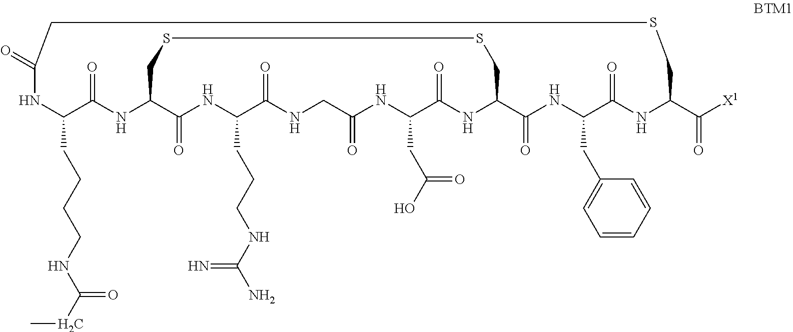

wherein X¹ is either —NH₂ or

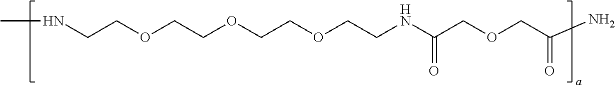

wherein a is an integer of from 1 to 10.

In Formula BTM1, a is preferably 1.

A preferred functionalised biological targeting molecule is of Formula IIIA:

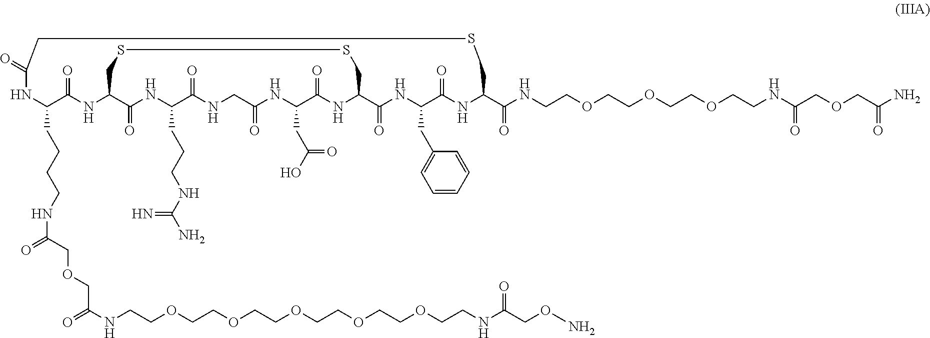

A preferred ¹⁸F-radiolabelled biological targeting molecule is ¹⁸F-fluciclatide of Formula (IVA):

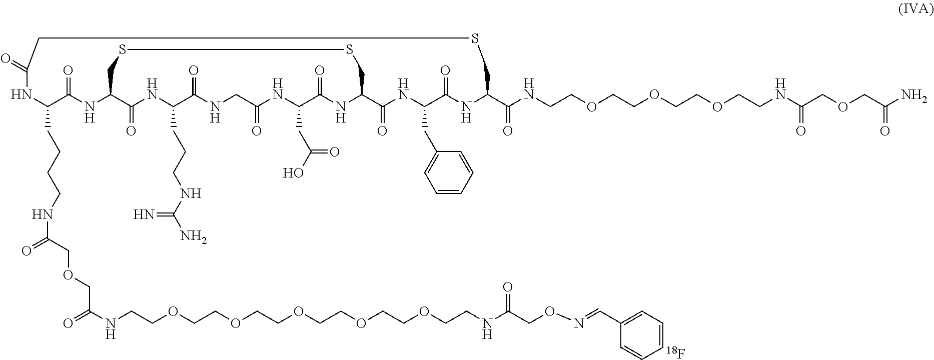

The c-Met binding peptide is preferably an 18 to 30-mer cyclic peptide of Formula V:

Z¹-[cMBP]-Z² (V)

where:
cMBP is of Formula II:

-(A)ⱼ-Q-(A′)ₖ- (II)

where Q is the amino acid sequence (SEQ-1):

-Cys^{a}-X^{1a}-Cys^{c}-X²-Gly-Pro-Pro -X³-Phe-Glu-Cys^{d}-Trp-Cys^{b}-Tyr-X⁴-X⁵-X⁶-
wherein X^{1a} is Asn, His or Tyr;
X² is Gly, Ser, Thr or Asn;
X³ is Thr or Arg;
X⁴ is Ala, Asp, Glu, Gly or Ser;
X⁵ is Ser or Thr;
X⁶ is Asp or Glu;
and Cys^{n-d} are each cysteine residues such that residues a and b as well as c and d are cyclised to form two separate disulfide bonds;
A and A′ are independently any amino acid other than Cys, with the proviso that at least one of A and A′ is present and is Lys;
j and k are independently integers of value 0 to 13, and are chosen such that [j+k]=1 to 13;
Z¹ is attached to the N-terminus of cMBP, and is H or M^{IG};
Z² is attached to the C-terminus of cMBP and is OH, OW, or M^{IG},
   where B^{C} is a biocompatible cation;
   each M^{IG} is independently a metabolism inhibiting group which is a biocompatible group which inhibits or suppresses in vivo metabolism of the cMBP peptide;
      wherein cMBP is labelled at the Lys residue of the A or A′ groups with ¹⁸F.

More preferably, the cMBP peptide is of Formula VA:

-(A)ⱼ-Q-(A′)_{z}-Lys- (VA)

wherein:
z is an integer of value 0 to 12, and [j+z]=0 to 12,
and cMBP comprises only one Lys residue.

In Formulae V and VA, Q preferably comprises the amino acid sequence of either SEQ-2 or SEQ-3:
Ser-Cys^{a}-X^{1a}-Cys^{c}-X²-Gly-Pro-Pro-X³-Phe-Glu-Cys^{d}-Trp-Cys^{b}-Tyr-X⁴-X⁵-X⁶ (SEQ-2);
Ala-Gly-Ser-Cys^{a}-X^{1a}-Cys^{c}-X²-Gly-Pro-Pro-X³-Phe-Glu-Cys^{d}-Trp-Cys^{b}-Tyr-X⁴-X⁵-X⁶-Gly-Thr (SEQ-3).

In Formulae V and VA, X³ is prcfcrably Arg.

The cMBP peptide most preferably has the amino acid sequence (SEQ-7): Ala-Gly-Ser-Cys^{a}-Tyr-Cys^{c}-Ser-Gly-Pro-Pro-Arg-Phe-Glu-Cys^{d}-Trp-Cys^{b}-Tyr-Glu-Thr-Glu-Gly-Thr-Gly-Gly-Gly-Lys.

The method of the second aspect is preferably carried out using an automated synthesizer apparatus, as described in the first aspect (above). Preferred aspects of the automated synthesis and automated synthesizer apparatus are as described in the first aspect (above).

The method of the second aspect is preferably carried out in a sterile manner, such that the ¹⁸F-radiolabelled biological targeting molecule is obtained as a radiopharmaceutical composition. The radiopharmaceutical composition comprises the ¹⁸F-radiolabelled biological targeting molecule, together with a ‘biocompatible carrier’ (as defined in the first aspect). Preferred aspects of the radiopharmaceutical composition and biocompatible carrier in the second aspect are as described for the first aspect (above).

When the radiopharmaceutical composition comprises ¹⁸F-fluciclatide of Formula (IVA), the composition preferably comprises a radioprotectant. Preferably, the radioprotectant is sodium 4-aminobenzoate (Na-pABA). A preferred concentration of Na-pABA to use is 1 to 3 mg/mL, preferably 1.5 to 2.5 mg/mL, most preferably about 2.0 mg/mL.

The method of the second aspect is preferably carried out in a sterile manner, such that a radiopharmaceutical composition is obtained. The radiopharmaceutical compositions of the present invention may be prepared by various methods:
(i) aseptic manufacture techniques in which the ¹⁸F-radiolabelling step is carried out in a clean room environment;
(ii) terminal sterilisation, in which the ¹⁸F-radiolabelling is carried out without using aseptic manufacture and then sterilised at the last step [eg. by gamma irradiation, autoclaving dry heat or chemical treatment (e.g. with ethylene oxide)];
(iii) kit methodology in which a sterile, non-radioactive kit formulation comprising a suitable precursor and optional excipients is reacted with a suitable supply of ¹⁸F;
(iv) aseptic manufacture techniques in which the ¹⁸F-radio labelling step is carried out using an automated synthesizer apparatus.

Method (iv) is preferred. Thus, the method of the second aspect is preferably carried out using an automated synthesizer apparatus.

By the term “automated synthesizer” is meant an automated module based on the principle of unit operations as described by Satyamurthy et al [Clin.Positr.Imag., 2(5), 233-253 (1999)]. The term ‘unit operations’ means that complex processes are reduced to a series of simple operations or reactions, which can be applied to a range of materials. Such automated synthesizers are preferred for the method of the present invention especially when a radiopharmaceutical composition is desired. They are commercially available from a range of suppliers [Satyamurthy et al, above], including: GE Healthcare; CTI Inc; Ion Beam Applications S.A. (Chemin du Cyclotron 3, B-1348 Louvain-La-Neuve, Belgium); Raytest (Germany) and Bioscan (USA).

Commercial automated synthesizers also provide suitable containers for the liquid radioactive waste generated as a result of the radiopharmaceutical preparation. Automated synthesizers are not typically provided with radiation shielding, since they are designed to be employed in a suitably configured radioactive work cell. The radioactive work cell provides suitable radiation shielding to protect the operator from potential radiation dose, as well as ventilation to remove chemical and/or radioactive vapours. The automated synthesizer preferably comprises a cassette. By the term “cassette” is meant a piece of apparatus designed to fit removably and interchangeably onto an automated synthesizer apparatus (as defined above), in such a way that mechanical movement of moving parts of the synthesizer controls the operation of the cassette from outside the cassette, i.e. externally. Suitable cassettes comprise a linear array of valves, each linked to a port where reagents or vials can be attached, by either needle puncture of an inverted septum-sealed vial, or by gas-tight, marrying joints. Each valve has a male-female joint which interfaces with a corresponding moving arm of the automated synthesizer. External rotation of the arm thus controls the opening or closing of the valve when the cassette is attached to the automated synthesizer. Additional moving parts of the automated synthesizer are designed to clip onto syringe plunger tips, and thus raise or depress syringe barrels.

The cassette is versatile, typically having several positions where reagents can be attached, and several suitable for attachment of syringe vials of reagents or chromatography cartridges (e.g. solid phase extraction or SPE). The cassette always comprises a reaction vessel. Such reaction vessels are preferably 0.5 to 10 mL, more preferably 0.5 to 5 mL and most preferably 0.5 to 4 mL in volume and are configured such that 3 or more ports of the cassette are connected thereto, to permit transfer of reagents or solvents from various ports on the cassette. Preferably the cassette has 15 to 40 valves in a linear array, most preferably 20 to 30, with 25 being especially preferred. The valves of the cassette are preferably each identical, and most preferably are 3-way valves. The cassettes are designed to be suitable for radiopharmaceutical manufacture and are therefore manufactured from materials which are of pharmaceutical grade and ideally also are resistant to radiolysis.

Preferred automated synthesizers of the present invention comprise a disposable or single use cassette which comprises all the reagents, reaction vessels and apparatus necessary to carry out the preparation of a given batch of radio fluorinated radiopharmaceutical. The cassette means that the automated synthesizer has the flexibility to be capable of making a variety of different radiopharmaceuticals with minimal risk of cross-contamination, by simply changing the cassette. The cassette approach also has the advantages of: simplified set-up hence reduced risk of operator error; improved GMP (Good Manufacturing Practice) compliance; multi-tracer capability; rapid change between production runs; pre-run automated diagnostic checking of the cassette and reagents; automated barcode cross-check of chemical reagents vs the synthesis to be carried out; reagent traceability; single-use and hence no risk of cross-contamination, tamper and abuse resistance.

In a third aspect, the present invention provides an ¹⁸F-labelled vinyl cyanide of Formula (II), (IIA), (IIB), (IIC) or (IID) as defined in the first aspect. The ¹⁸F-labelled vinyl cyanide of the third aspect is preferably of Formula HA or IID, more preferably of Formula IIB, most preferably of Formula IIC.

The ¹⁸F-labelled vinyl cyanide of the third aspect may be obtained by condensation of the aldehyde of interest in acetonitrile under basic conditions at a temperature of 50 to 80° C.

In a fourth aspect, the present invention provides a radiopharmaceutical composition which comprises:
(i) an ¹⁸F-radiolabelled biological targeting molecule of Formula IV:

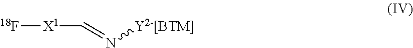

(ii) an ¹⁸F-labelled vinyl cyanide of Formula (II):

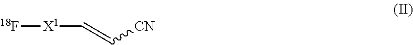

wherein Y² and BTM are as defined in the second aspect, and
X¹ is as defined in the first aspect;
together with a biocompatible carrier, in a form suitable for mammalian administration;
wherein the molar ratio of IV:II is at least 10:1.

Preferred embodiments of X¹ in the third aspect are as described in the first aspect (above). Preferred embodiments of BTM in the third aspect are as described in the second aspect (above).

The ‘biocompatible carrier’ and preferred embodiments thereof in the fourth aspect, are as defined in the first aspect (above). In this fourth aspect, the biocompatible carrier may optionally include acetonitrile.

Amino-oxy functionalised peptides can be prepared by the methods of Poethko et al [J.Nucl.Med., 45, 892-902 (2004)], Schirrmacher et al [Bioconj.Chem., 18, 2085-2089 (2007)], Solbakken et al [Bioorg.Med.Chem.Lett, 16, 6190-6193 (2006)] or Glaser et al [Bioconj. Chem., 19, 951-957 (2008)]. The amino-oxy group may optionally be conjugated in two steps. First, the corresponding N-protected amino-oxy carboxylic acid or N-protected amino-oxy activated ester is conjugated to the peptide. Second, the intermediate N-protected amino-oxy functionalised peptide is deprotected to give the desired product (see Solbakken and Glaser cited above). N-protected amino-oxy carboxylic acids such as Boc-NH—O—CH₂(C═O)OH and Eei-N—O—CH₂(C═O)OH are commercially available, e.g. from Novabiochem and IRIS. The term “protected” refers to the use of a protecting group. The term “protecting group” has its conventional meaning, and refers to a group which inhibits or suppresses undesirable chemical reactions, but which is designed to be sufficiently reactive that it may be cleaved from the functional group in question under mild enough conditions that do not modify the rest of the molecule. After deprotection the desired product is obtained. Amine protecting groups are well known to those skilled in the art and are suitably chosen from: Boc (where Boc is tert-butyloxycarbonyl); Eei (where Eei is ethoxyethylidene); Fmoc (where Fmoc is fluorenylmethoxycarbonyl); trifluoroacetyl; allyloxycarbonyl; Dde [i.e. 1-(4,4-dimethyl-2,6-dioxocyclohexylidene)ethyl] or Npys (i.e. 3-nitro-2-pyridine sulfenyl). The use of further protecting groups are described in Protective Groups in Organic Synthesis, 4^{th} Edition, Theorodora W. Greene and Peter G. M. Wuts, [Wiley Blackwell, (2006)]. Preferred amine protecting groups are Boc and Eei, most preferably Eei.

The precursor to [¹⁸F]-fluorobenzaldehyde, i.e. Me₃N⁺—C₆H₄-CHO. CF₃SO₃⁻ is obtained by the method of Haka et al [J.Lab.Comp.Radiopharm., 27, 823-833 (1989)].

The ¹⁸F-aldehyde [¹⁸F]-FBPA can be prepared by the method of Carberry et al [Bioconj.Chem., 22, 642-653 (2011) and Bioorg.Med.Chem.Lett., 21, 6992-6995 (2011)]:

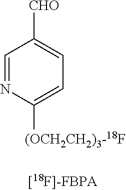

Other peptides can be obtained by solid phase peptide synthesis as described in P. Lloyd-Williams, F. Albericio and E. Girald; Chemical Approaches to the Synthesis of Peptides and Proteins, CRC Press, 1997.

In a fifth aspect, the present invention provides a method of imaging the human or animal body which comprises generating a PET image of at least a part of said body to which the radiopharmaceutical composition of the fourth aspect has distributed.

Preferred aspects of the radiopharmaceutical composition and the ¹⁸F-labelled BTM therein in the fifth aspect are as described in the fourth and second aspects of the present invention respectively (see above).

When the BTM targets of the integrin αᵥβ₃ receptor, the method of the fifth aspect is preferably carried out where the part of the body is disease state where abnormal expression of the integrin αᵥβ₃ receptor is involved, in particular angiogenesis. Such disease states include rheumatoid arthritis, psoriasis, restenosis, retinopathy and tumour growth. A preferred such disease state of the fifth aspect is tumour growth. Positron Emission Tomography (PET) imaging of integrin αᵥβ₃ expression is described by Beer et al [Theranostics, 1, 48-57 (2011)].

The imaging method of the fifth aspect may optionally be carried out repeatedly to monitor the effect of treatment of a human or animal body with a drug, said imaging being effected before and after treatment with said drug, and optionally also during treatment with said drug. Of particular interest is early monitoring of the efficacy of anti-angiogenic cancer therapy to ensure that malignant growth is controlled before the condition becomes terminal. Such therapy monitoring imaging is described by Battle et al [J.Nucl.Med., 52(3), 424-430 (2011)] and Morrison et al [J.Nucl.Med., 50(1), 116-122 (2009) and Theranostics, 1, 149-153 (2011)].

The method of the fifth aspect is preferably carried out whereby the radiopharmaceutical composition has been previously administered to the mammalian body. By “previously administered” is meant that the step involving the clinician, wherein the imaging agent is given to the patient e.g. as an intravenous injection, has already been carried out prior to imaging.

In a sixth aspect, the present invention provides a method of diagnosis of the human or animal body which comprises the imaging method of the fifth aspect.

Preferred aspects of the radiopharmaceutical composition and ¹⁸F-BTM in the sixth aspect are as described in the fourth and second aspects (above).

The invention is illustrated by the non-limiting Examples detailed below. Example 1 provides the synthesis of Precursor 1 of the invention. Example 2 provides the synthesis of [^{18]}F-FBA, and Example 3 the purification of [¹⁸F]-FBA to obtain compositions of the invention. Example 4 provides the synthesis of Compound 1 of the invention using the purified [¹⁸F]-FBA composition of the invention. Example 5 provides experimental evidence of the formation of cyanovinyl species under mild conditions on reaction with a non-radioactive benzaldehyde derivative, and their characterisation. Example 6 shows that [¹⁸F]-FBA readily undergoes reaction with acetonitrile, to more analogous cyanovinyl species.

Abbreviations.

Conventional single letter or 3-letter amino acid abbreviations are used.

Ac: Acetyl.

ACN: Acetonitrile.

BTM: biological targeting moiety.

Boc: tert-Butyloxycarbonyl.

DIPEA: N,N-diisopropylethylamine.

DMAB: 4-(dimethylamino)benzaldehyde.

DMSO: Dimethylsulfoxide,

EOS: End of synthesis.

FBA: 4-Fluorobenzaldehyde.

Fmoc: 9-Fluorenylmethoxycarbonyl.

HATU: O-(7-Azabenzotriazol-1-yl)-N,N,N′,N′-tetramethyluronium hexafluorophosphate.

HPLC: High performance liquid chromatography.

LC-UV: Liquid Chromatography with ultraviolet detection.

MCX Mixed mode cation exchange cartridge

NMM: N-methymorpholine.

NMP: 1-Methyl-2-pyrrolidinone.

PBS: Phosphate-buffered saline.

PyBOP: Benzotriazol-1-yl-oxytripyrrolidinophosphonium hexafluorophosphate.

RAC: radioactive concentration.

RCP: Radiochemical purity.

RT: room temperature.

SPE: solid-phase extraction.

tBu: tert-Butyl.

TFA: Trifluoroacetic acid.

TFP: Tetrafluorophenyl.

TMAB: 4-(trimethylammonium)benzaldehyde.

T_{R}: retention time.

TABLE 1
Compounds of the Invention.

| Name | Structure |
|---|---|
| Pep- tide 1 | 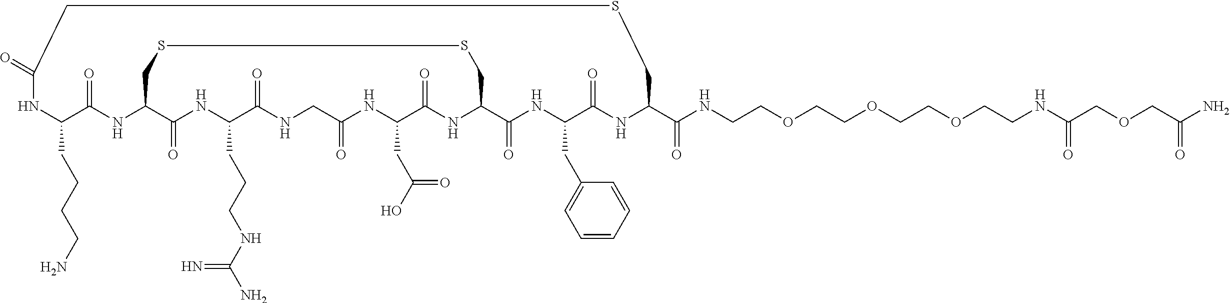 |
| Pre- cur- sor 1 | 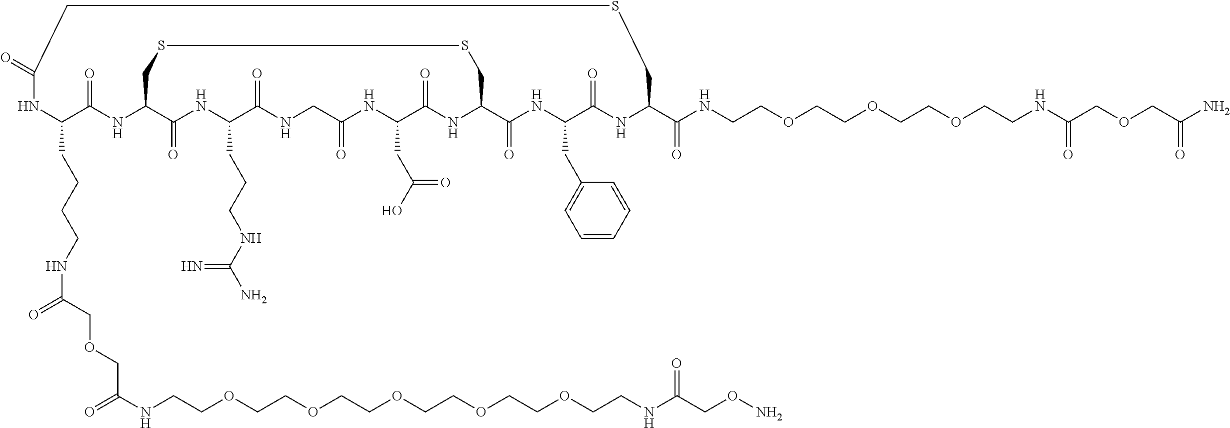 |
| Com- pound 1 | 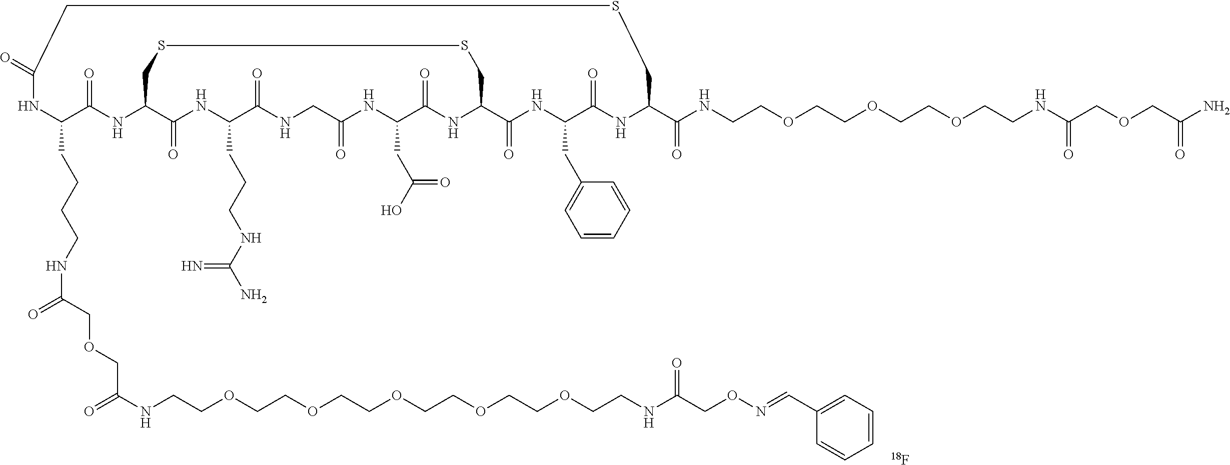 |

### EXAMPLE 1

### Synthesis of Precursor 1

Peptide 1 was synthesised using standard peptide synthesis.

### (a) 1,17-Diazido-3,6,9,12,15-pentaoxaheptadecane

A solution of dry hexaethylene glycol (25 g, 88 mmol) and methanesulfonyl chloride (22.3 g, 195 mmol) in dry THF (125 mL) was kept under argon and cooled to 0° C. in an ice/water bath. A solution of triethylamine (19.7 g, 195 mmol) in dry THF (25 mL) was added dropwise over 45 min. After 1 hr the cooling bath was removed and the reaction was stirred for another for 4 hrs. Water (55 mL) was then added to the mixture, followed by sodium hydrogencarbonate (5.3 g, to pH 8) and sodium azide (12.7 g, 195 mmol). THF was removed by distillation and the aqueous solution was refluxed for 24 h (two layers were formed). The mixture was cooled, ether (100 mL) was added and the aqueous phase was saturated with sodium chloride. The phases were separated and the aqueous phase was extracted with ether (4×50 mL). The combined organic phases were washed with brine (2×50 mL) and dried (MgSO₄).

Filtration and evaporation of the solvent gave a yellow oil 26 g (89%). The product was used in the next step without further purification.

### (b) 17-Azido-3,6,9,12,15-pentaoxaheptadecanamine

To a vigorously stirred suspension of 1,17-diazido-3,6,9,12,15-pentaoxaheptadecane (25 g, 75 mmol) in 5% HCl (200 mL) was added a solution of triphenylphosphine (19.2 g, 73 mmol) in ether (150 mL) over 3 hrs at room temperature. The reaction mixture was stirred for additional 24 hrs. The phases were separated and the aqueous phase was extracted with dichloromethane (3×40 mL). The aqueous phase was cooled in an ice/water bath and the pH was adjusted to 12 by addition of solid potassium hydroxide. The aqueous phase was concentrated and the product was taken up in dichloromethane (150 mL). The organic phase was dried (Na₂SO₄) and concentrated giving a yellow oil 22 g (95%). The product was identified by electrospray mass spectrometry (ESI-MS) (MH+calculated: 307.19; found 307.4). The crude oil was used in the next step without further purification.

### (c) 23-Azido-5-oxo-6-aza-3,9,12,15,18,21-hexaoxatricosano is acid

To a solution of 17-azido-3,6,9,12,15-pentaoxaheptadecanamine (15 g, 50 mmol) in dichloromethane (100 mL) was added diglycolic anhydride (Acros, 6.4 g, 55 mmol).

The reaction mixture was stirred overnight. The reaction was monitored by ESI-MS analysis, and more reagents were added to drive the reaction to completion. The solution was concentrated to give a yellow residue which was dissolved in water (250 mL). The product was isolated from the aqueous phase by continuous extraction with dichloromethane overnight. Drying and evaporation of the solvent gave a yield of 18 g (85%). The product was characterized by ESI-MS analysis (MH+calculated: 423.20; found 423.4). The product was used in the next step without further purification.

### (d) 23-Amino-5-oxo-6-aza-3 ,9,12,15,18,21-hexaoxatricosanoic acid

23-Azido-5-oxo-6-aza-3,9,12,15,18,21-hexaoxatricosanoic acid (9.0 g, 21 mmol) was dissolved in water (50 mL) and reduced using H₂(g)-Pd/C (10%). The reaction was run until ESI-MS analysis showed complete conversion to the desired product (MH+calculated: 397.2; found 397.6). The crude product was used in the next step without further purification.

### (e) (Boc-aminooxy)acetyl-PEG(6)-diglycolic acid

A solution of dicyclohexycarbodiimide (515 mg, 2.50 mmol) in dioxan (2.5 mL) was added dropwise to a solution of (Boc-aminooxy)acetic acid (477 mg, 2.50 mmol) and N-hydroxysuccinimide (287 mg, 2.50 mmol) in dioxan (2.5 mL). The reaction was stirred at RT for lh and filtered. The filtrate was transferred to a reaction vessel containing a solution of 23-amino-5-oxo-6-aza-3,9,12,15,18,21-hexaoxatricosanoic acid (1.0 g, 2.5 mmol) and NMM (278 μl, 2.50 mmol) in water (5 mL). The mixture was stirred at RT for 30 min. ESI-MS analysis showed complete conversion to the desired product (MH+calculated: 570.28; found 570.6). The crude product was purified by preparative HPLC (column: Phenomenex Luna 5p. C18 (2) 250×21.20 mm, detection: 214 nm, gradient: 0-50% B over 60 min where A=H₂O/0.1% TFA and B=acetonitrile/0.1% TFA, flow rate: 10 mL/min) affording 500 mg (38%) of pure product. The product was analyzed by HPLC (column: Phenomenex Luna 3μ C18 (2), 50×2.00 mm, detection: 214 nm, gradient: 0-50% B over 10 min where A=H₂O/0.1% TFA and B=acetonitrile/0.1% TFA, flow rate: 0.75 mL/min, Rt=5.52 min). Further confirmation was carried out by NMR analysis.

### (f) Conjugation of (Boc-aminooxy)acetyl-PEG(6)-diglycolic acid to Peptide 1

(Boc-aminooxy)acetyl-PEG(6)-diglycotic acid (0.15 mmol, 85 mg) and PyAOP (0.13 mmol, 68 mg) were dissolved in DMF (2 mL). NMM (0.20 mmol, 20 μL) was added and the mixture was stirred for 10 min. A solution of Peptide 1 (0.100 mmol, 126 mg) and NMM (0.20 mmol, 20 μL) in DMF (4 mL) was added and the reaction mixture was stirred for 25 min. Additional NMM (0.20 mmol, 20 μl) was added and the mixture was stirred for another 15 min. DMF was evaporated in vacuo and the product was taken up in 10% acetonitrile-water and purified by preparative HPLC (column: Phenomenex Luna 5g C18 (2) 250×21.20 mm, detection: UV 214 nm, gradient: 5-50% B over 40 min where A=H₂O/0.1% TFA and B=acetonitrile/0.1% TFA, flow rate: 10 mL/min,) affording 100 mg semi-pure product. A second purification step where TFA was replaced by HCOOH (gradient: 0-30% B, otherwise same conditions as above) afforded 89 mg (50%). The product was analysed by HPLC (column: Phenomenex Luna 3μ C18 (2) 50×2 mm, detection: UV 214 nm, gradient: 0-30% B over 10 min where A=H₂O/0.1% HCOOH and B=acetonitrile/0.1% HCOOH, flow rate: 0.3 mL/min, Rt: 10.21 min). Further product characterisation was carried out using ESI-MS (MH22+calculated: 905.4, found: 906.0).

### (g) Deprotection

Deprotection was carried out by addition of TFA containing 5% water to 10 mg of peptide.

### EXAMPLE 2

### Radiosynthesis of ¹⁸F-benzaldehyde (¹⁸F-FBA)

[¹⁸F]-fluoride was produced using a GEMS PETtrace cyclotron with a silver target via the [¹⁸O](p,n) [¹⁸F] nuclear reaction. Total target volumes of 1.5-3.5 mL were used. The radiofluoride was trapped on a Waters QMA cartridge (pre-conditioned with carbonate), and the fluoride is eluted with a solution of Kryptofix_{2.2.2}. (4 mg, 10.7 μM) and potassium carbonate (0.56 mg, 4.1 μM) in water (80 μL) and acetonitrile (320 μL). Nitrogen was used to drive the solution off the QMA cartridge to the reaction vessel. The [¹⁸F]-fluoride was dried for 9 minutes at 120° C. under a steady stream of nitrogen and vacuum. Trimethylammonium benzaldehyde triflate, [Haka et al, J. Lab. Comp. Radiopharm., 27, 823-833 (1989)] (3.3 mg, 10.5 μM), in DMSO (1.1 mL) was added to the dried [¹⁸F]-fluoride, and the mixture heated at 105° C. for 7 minutes to produce 4-[¹⁸F]-fluorobenzaldehyde.

### EXAMPLE 3

### Purification of ¹⁸F-Fluorobenzaldehyde (¹⁸F-FBA)

The crude labelling mixture from Example 2 was diluted with ammonium hydroxide solution and loaded onto an MCX+SPE cartridge (pre-conditioned with water as part of the FASTlab sequence). The cartridge was washed with water, dried with nitrogen gas before elution of 4-[¹⁸F]-fluorobenzaldehyde back to the reaction vessel in ethanol (1.8 mL). A total volume of ethanol of 2.2 mL was used for the elution but the initial portion (0.4 mL) was discarded as this did not contain [¹⁸F]-FBA. 4-7% (decay corrected) of the [¹⁸F] radioactivity remained trapped on the cartridge.

The temperature and time of the [¹⁸F]-FBA-labelling step were selected to minimise the cyanovinyl species formation compromising the FBA yield. The cyanovinyl species formation was also minimized as a consequence of optimizing the [¹⁸F]-fluoride drying step, to remove acetonitrile.

### EXAMPLE 4

### Preparation of [¹⁸F]-fluciclatide (Compound 1)

The conjugation of [¹⁸F]-FBA with Precursor 1 (5 mg) was performed in a solution of ethanol (1.8 mL) and water (1.8 mL) in the presence of aniline hydrochloride. The reaction mixture was maintained at 60° C. for 5 minutes.

### EXAMPLE 5

### Reaction of 4-(Trimethylammonium)benzaldehyde (TMAB) with Acetonitrile).

Two experiments were carried out:

(A) TMAB was mixed with CH₃CN, K₂CO₃ and Kryptofix 222 in DMSO;

(B) TMAB was mixed with CD₃CN, K₂CO₃ and Kryptofix 222 in DMSO. An excess of

¹⁹F-FBA was also added.

The reaction products from Experiments A and B were analysed using LC-UV/MS. An unknown peak in the (A) chromatogram was analysed by MS, and shown to have a base peak at m/z 187.1. The corresponding peak in the (B) chromatogram whens analysed by MS, had a base peak at m/z 188.1. That corresponds to the reaction shown (for the CD₃CN reaction):

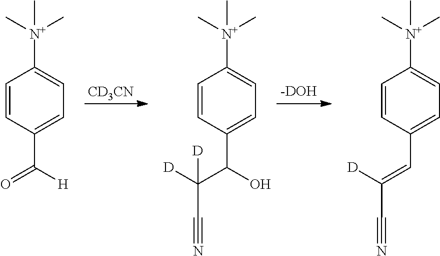

| Exact Mass | 164.1075 | 207.1464 | 188.1297 |
|---|---|---|---|
| Molecular | C₁₀H₁₄NO | C₁₂H₁₅N₂OD₂ | C₁₂H₁₄N₂D |
| formula | | | |

### EXAMPLE 6

### Reaction of 4-Fluorobenzaldehyde (FBA) with Acetonitrile)

¹⁹F-FBA was used. FBA was mixed with CH₃CN, K₂CO₃ and Kryptofix 222 in DMSO. FBA has little or no MS response, so data corresponding to that of Example 5 was not feasible. LC-UV showed, however, that no FBA was left in the sample, and that a new major peak formed with a later elution time than FBA.

The cyanovinyl adducts of Example 5 showed a shift in X. of ca. 26 nm to higher wavelength compared to TMAB. A similar shift was observed here for the later-eluting reaction product—hence that was ascribed to a cyanovinyl species also.

1. An ¹⁸F-labelled aldehyde composition which comprises an ¹⁸F-labelled aldehyde of Formula (I) and an ¹⁸F-labelled vinyl cyanide of Formula (II): 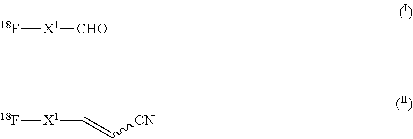
where X¹ is the same in Formulae (I) and (II), and is a C₄₋₁₆ bivalent organic radical;
and wherein (i) the molar ratio of I:II is at least 10:1; and
(ii) acetonitrile is excluded from said composition.

2. The ¹⁸F-labelled aldehyde composition of claim 1, where the ¹⁸F-labelled aldehyde is of Formula (IA) and the ¹⁸F-labelled vinyl cyanide is of Formula (IIA): 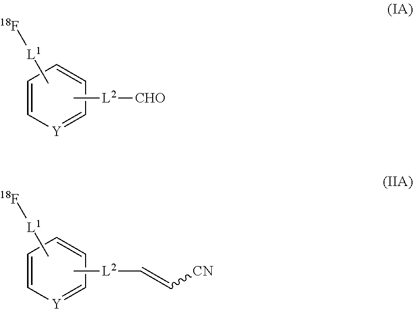
where:
Y is independently C or N;
L¹ and L² are independently linker groups chosen from —(CH₂)ₓ—, —O—(CH₂)_{y}— or —(OCH₂CH₂)_{y}—; and
wherein x is independently an integer of value 0 to 3, and y is independently an integer of value 2 to 4.

3. The ¹⁸F-labelled aldehyde composition of claim 2, where the ¹⁸F-labelled aldehyde is of Formula (IB) and the ¹⁸F-labelled vinyl cyanide is of Formula (IIB): 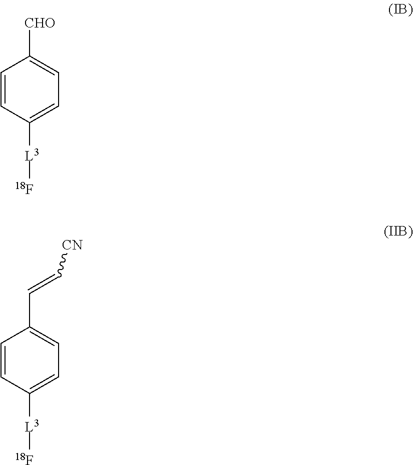
where:
L³ is —(CH₂)ₓ— or —O—(CH₂)_{y}—, and x and y are as defined in claim 2.

4. The ¹⁸F-labelled aldehyde composition of claim 3, where the ¹⁸F-labelled aldehyde is of Formula (IC) and the ¹⁸F-labelled vinyl cyanide is of Formula (IIC):

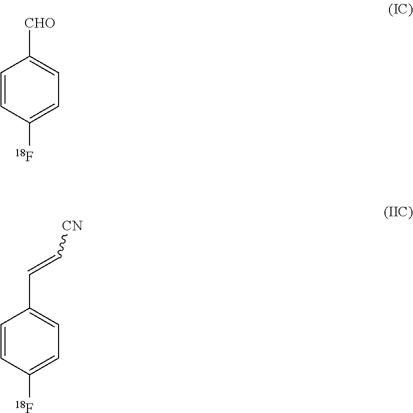 [SMILES: O=Cc1ccc([18F])cc1 ; N#CC=Cc1ccc([18F])cc1]

5. The ¹⁸F-labelled aldehyde composition of claim 2, where the ¹⁸F-labelled aldehyde is of Formula (ID) and the ¹⁸F-labelled vinyl cyanide is of Formula (IID): 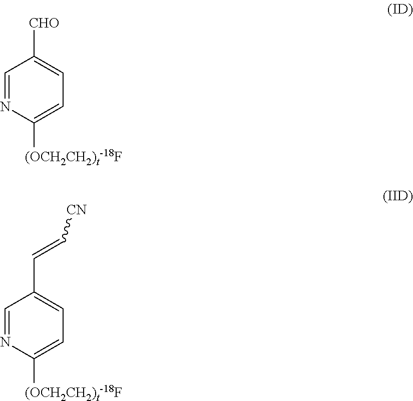
wherein t is an integer of value 1 to 3.

6. The ¹⁸F-labelled aldehyde composition of claim 1, which is provided in a water miscible organic solvent or an aqueous mixture thereof.

7. The ¹⁸F-labelled aldehyde composition of claim 6, which is a radiopharmaceutical composition which comprises the ¹⁸F-labelled aldehyde composition together with a biocompatible carrier, in a form suitable for mammalian administration.

8. A method of ¹⁸F-radiolabelling a biological targeting molecule, which comprises:
(i) provision of the ¹⁸F-labelled aldehyde composition of claim 1;
(ii) provision of a functionalised biological targeting molecule of Formula III:
Y¹-[BTM] (III)
wherein Y¹ is —NH₂ or —O—NH₂;
(iii) reaction of the composition from step (i) with Y¹-[BTM] from step (ii) to give the ¹⁸F-radiolabelled biological targeting molecule of Formula IV: 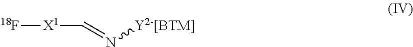
wherein Y² is absent or is —O—.

9. The method of claim 8, where the ¹⁸F-labelled aldehyde composition is as defined in claim 2 any one of claims 2 to 7.

10. The method of claim 8 or claim 9, where the BTM comprises a single amino acid, a 3-100 mer peptide, an enzyme substrate, an enzyme antagonist an enzyme agonist, an enzyme inhibitor, or a receptor-binding compound.

11. The method of claim 8 any one of claims 8 to 10, where the BTM comprises an RGD peptide.

12. The method of claim 11, where the functionalised biological targeting molecule is of Formula IIIA:

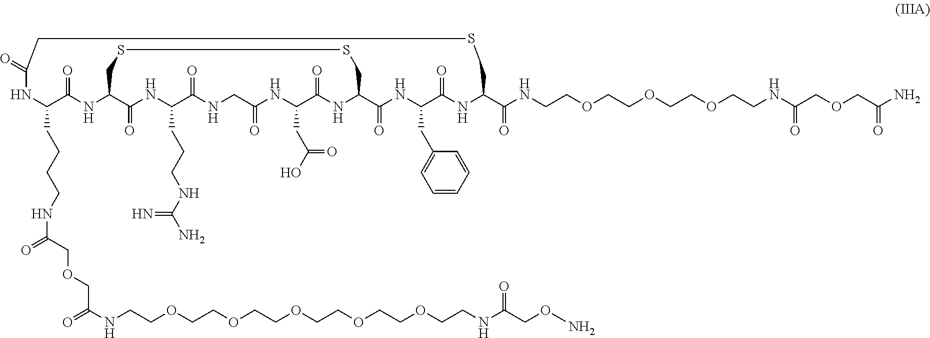

13. The method of claim 8, where the ¹⁸F-radiolabelled biological targeting molecule is of Formula (IVA):

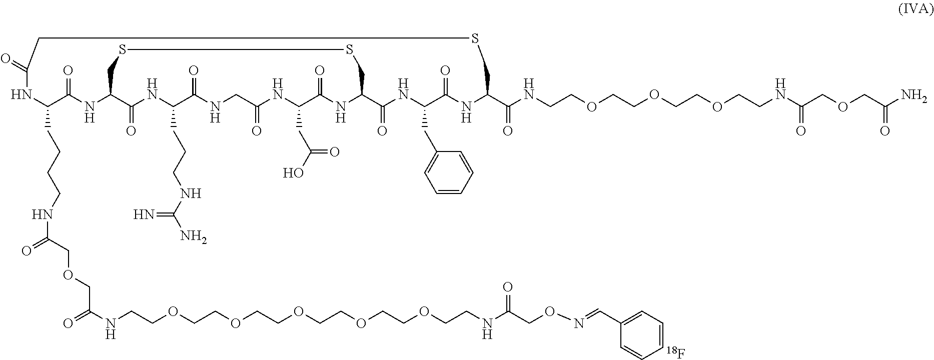

14. The method of claim 8, which is carried out using an automated synthesizer apparatus.

15. The method of claim 14, which is carried out in a sterile manner, such that the ¹⁸F-radiolabelled biological targeting molecule is obtained as a radiopharmaceutical composition.

16. An ¹⁸F-labelled vinyl cyanide of Formula (II), (IIA), (IIB), (IIC) or (IID) as defined in claim 1 any one of claims 1 to 5.

17. A radiopharmaceutical composition which comprises:
(i) an ¹⁸F-radiolabelled biological targeting molecule of Formula IV: 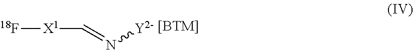
(ii) an ¹⁸F-labelled vinyl cyanide of Formula (II): 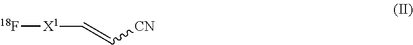
wherein Y² and BTM are as defined in claim 8, and X¹ is as defined in claim 1;
together with a biocompatible carrier, in a form suitable for mammalian administration;
wherein the molar ratio of IV:II is at least 10:1.

18. The radiopharmaceutical composition of claim 17, where X¹ is as defined in claim 2 any one of claims 2 to 5.

19. The radiopharmaceutical composition of claim 17 or claim 18, where the BTM is as defined in claim 10.

20. A method of imaging the human or animal body which comprises generating a PET image of at least a part of said body to which the radiopharmaceutical composition of claim 17 has been distributed.

21.-23. (canceled)
